(19) United States
(12) Patent Application Publication
(10) Pub. No.: US 2012/0277465 A1
(43) Pub. Date: Nov. 1, 2012

(54) REDUCTION OF CARBON DIOXIDE TO CARBOXYLIC ACIDS, GLYCOLS, AND CARBOXYLATES

(75) Inventors: Emily Barton Cole, Princeton, NJ (US); Kyle Teamey, Washington, DC (US); Andrew B. Bocarsly, Plainsboro, NJ (US); Narayanappa Sivasankar, Plainsboro, NJ (US)

(73) Assignee: LIQUID LIGHT, INC., Monmouth Junction, NJ (US)

(21) Appl. No.: 13/542,152

(22) Filed: Jul. 5, 2012

Related U.S. Application Data
(63) Continuation-in-part of application No. 12/846,221, filed on Jul. 29, 2010.
(60) Provisional application No. 61/504,848, filed on Jul. 6, 2011.

(51) Int. Cl. C07C 27/04; C07C 51/347; C07C 29/149; C07C 45/41; C07C 51/00

(52) U.S. Cl. 562/577; 568/484; 562/609; 568/884; 562/579; 568/864; 562/607; 562/589; 562/592; 422/187

(57) ABSTRACT

Methods and systems for electrochemical conversion of carbon dioxide to carboxylic acids, glycols, and carboxylates are disclosed. A method may include, but is not limited to, steps (A) to (D). Step (A) may introduce water to a first compartment of an electrochemical cell. The first compartment may include an anode. Step (B) may introduce carbon dioxide to a second compartment of the electrochemical cell. The second compartment may include a solution of an electrolyte and a cathode. Step (C) may apply an electrical potential between the anode and the cathode in the electrochemical cell sufficient to reduce the carbon dioxide to a carboxylic acid intermediate. Step (D) may contact the carboxylic acid intermediate with hydrogen to produce a reaction product.

### CROSS-REFERENCE TO RELATED APPLICATIONS

The present application claims the benefit under 35 U.S.C. §119(e) of U.S. Patent Application Ser. No. 61/504,848, filed Jul. 6, 2011.

The present application claims the benefit under 35 U.S.C. §120 of U.S. patent application Ser. No. 12/846,221, filed Jul. 29, 2010.

The above-listed applications are hereby incorporated by reference in their entirety.

### FIELD

The present disclosure generally relates to the field of electrochemical reactions, and more particularly to methods and/or systems for electrochemical production of carboxylic acids, glycols, and carboxylates from carbon dioxide.

### BACKGROUND

The combustion of fossil fuels in activities such as electricity generation, transportation, and manufacturing produces billions of tons of carbon dioxide annually. Research since the 1970s indicates increasing concentrations of carbon dioxide in the atmosphere may be responsible for altering the Earth's climate, changing the pH of the ocean and other potentially damaging effects. Countries around the world, including the United States, are seeking ways to mitigate emissions of carbon dioxide.

A mechanism for mitigating emissions is to convert carbon dioxide into economically valuable materials such as fuels and industrial chemicals. If the carbon dioxide is converted using energy from renewable sources, both mitigation of carbon dioxide emissions and conversion of renewable energy into a chemical form that can be stored for later use may be possible.

### SUMMARY OF THE PREFERRED EMBODIMENTS

The present invention is directed to using particular cathode materials, homogenous heterocyclic amine catalysts, and an electrolytic solution to reduce carbon dioxide to a carboxylic acid intermediate preferably including at least one of formic acid, glycolic acid, glyoxylic acid, oxalic acid, or lactic acid. The carboxylic acid intermediate may be processed further to yield a glycol-based reaction product. The present invention includes the process, system, and various components thereof.

It is to be understood that both the foregoing general description and the following detailed description are exemplary and explanatory only and are not necessarily restrictive of the disclosure as claimed. The accompanying drawings, which are incorporated in and constitute a part of the specification, illustrate an embodiment of the disclosure and together with the general description, serve to explain the principles of the disclosure.

### BRIEF DESCRIPTION OF THE DRAWINGS

The numerous advantages of the present disclosure may be better understood by those skilled in the art by reference to the accompanying figures in which:

FIGS. 1A and 1B depict a block diagram of a preferred system in accordance with an embodiment of the present disclosure;

FIG. 2 is a flow diagram of a preferred method of electrochemical production of a reaction product from carbon dioxide; and

FIG. 3 is a flow diagram of another preferred method of electrochemical production of a reaction product from carbon dioxide.

### DETAILED DESCRIPTION OF THE PREFERRED EMBODIMENTS

Reference will now be made in detail to the presently preferred embodiments of the present disclosure, examples of which are illustrated in the accompanying drawings.

In accordance with some embodiments of the present disclosure, an electrochemical system is provided that converts carbon dioxide to carboxylic acid intermediates, carboxylic acids, and glycols. Use of a homogenous heterocyclic catalyst facilitates the process.

Before any embodiments of the invention are explained in detail, it is to be understood that the embodiments described below do not limit the scope of the claims that follow. Also, it is to be understood that the phraseology and terminology used herein is for the purpose of description and should not be regarded as limiting. The use of terms such as “including,” “comprising,” or “having” and variations thereof herein are generally meant to encompass the item listed thereafter and equivalents thereof as well as additional items. Further, unless otherwise noted, technical terms may be used according to conventional usage.

In certain preferred embodiments, the reduction of the carbon dioxide to produce carboxylic acid intermediates, carboxylic acids, and glycols may be preferably achieved in a divided electrochemical or photoelectrochemical cell having at least two compartments. One compartment contains an anode suitable to oxidize water, and another compartment contains a working cathode electrode and a homogenous heterocyclic amine catalyst. The compartments may be separated by a porous glass frit, microporous separator, ion exchange membrane, or other ion conducting bridge. Both compartments generally contain an aqueous solution of an electrolyte. Carbon dioxide gas may be continuously bubbled through the cathodic electrolyte solution to preferably saturate the solution or the solution may be pre-saturated with carbon dioxide.

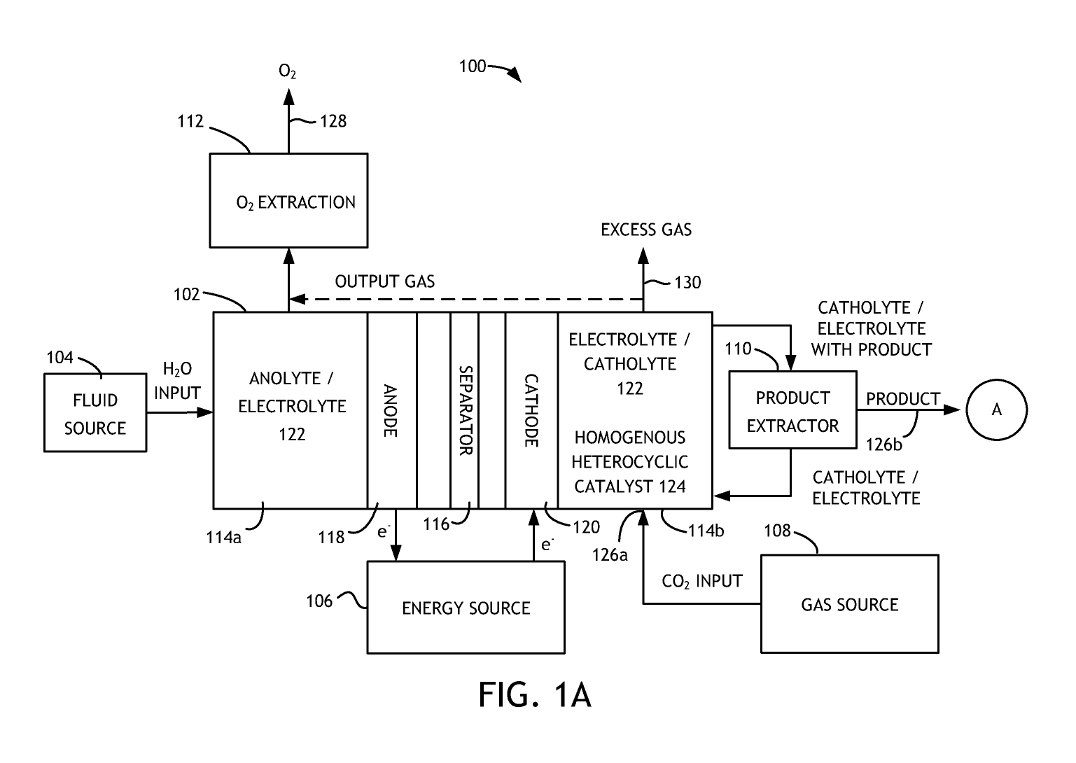

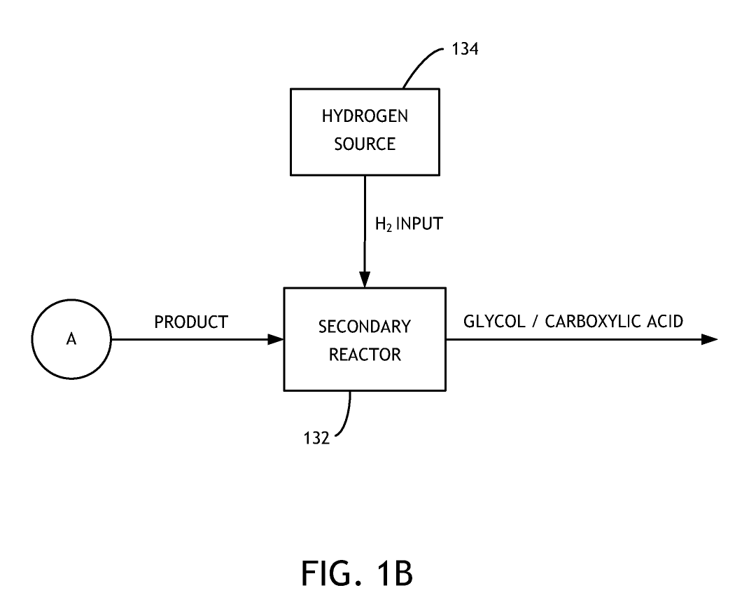

Referring to FIG. 1, a block diagram of a system 100 is shown in accordance with an embodiment of the present invention. System 100 may be utilized for electrochemical production of carboxylic acid intermediates, carboxylic acids, and glycols from carbon dioxide and water (and hydrogen for glycol production). The system (or apparatus) 100 generally comprises a cell (or container) 102, a liquid source 104 (preferably a water source, but may include an organic solvent source), an energy source 106, a gas source 108 (preferably a carbon dioxide source), a product extractor 110 and an oxygen extractor 112. A product or product mixture may be output from the product extractor 110 after extraction. An output gas containing oxygen may be output from the oxygen extractor 112 after extraction.

The cell 102 may be implemented as a divided cell. The divided cell may be a divided electrochemical cell and/or a divided photochemical cell. The cell 102 is generally operational to reduce carbon dioxide (CO₂) into products or product intermediates. In particular implementations, the cell 102 is operational to reduce carbon dioxide to carboxylic acid intermediates (including salts such as formate, glycolate, glyoxylate, oxalate, and lactate), carboxylic acids, and glycols. The reduction generally takes place by introducing (e.g., bubbling) carbon dioxide into an electrolyte solution in the cell 102. A cathode 120 in the cell 102 may reduce the carbon dioxide into a carboxylic acid or a carboxylic acid intermediate. The production of a carboxylic acid or carboxylic acid intermediate may be dependent on the pH of the electrolyte solution, with lower pH ranges favoring carboxylic acid production. The pH of the cathode compartment may be adjusted to favor production of one of a carboxylic acid or carboxylic acid intermediate over production of the other, such as by introducing an acid (e.g., HCl or H₂SO₄) to the cathode compartment. Hydrogen may be introduced to the carboxylic acid or carboxylic acid intermediate to produce a glycol or a carboxylic acid, respectively. The hydrogen may be derived from natural gas or water.

The cell 102 generally comprises two or more compartments (or chambers) 114a-114b, a separator (or membrane) 116, an anode 118, and a cathode 120. The anode 118 may be disposed in a given compartment (e.g., 114a). The cathode 120 may be disposed in another compartment (e.g., 114b) on an opposite side of the separator 116 as the anode 118. In particular implementations, the cathode 120 includes materials suitable for the reduction of carbon dioxide including cadmium, a cadmium alloy, cobalt, a cobalt alloy, nickel, a nickel alloy, chromium, a chromium alloy, indium, an indium alloy, iron, an iron alloy, copper, a copper alloy, lead, a lead alloy, palladium, a palladium alloy, platinum, a platinum alloy, molybdenum, a molybdenum alloy, tungsten, a tungsten alloy, niobium, a niobium alloy, silver, a silver alloy, tin, a tin alloy, rhodium, a rhodium alloy, ruthenium, a ruthenium alloy, carbon, and mixtures thereof. An electrolyte solution 122 (e.g., anolyte or catholyte 122) may fill both compartments 114a-114b. The aqueous solution 122 preferably includes water as a solvent and water soluble salts for providing various cations and anions in solution, however an organic solvent may also be utilized. In certain implementations, the organic solvent is present in an aqueous solution, whereas in other implementations the organic solvent is present in a non-aqueous solution. The catholyte 122 may include sodium and/or potassium cations or a quaternary amine (preferably tetramethyl ammonium or tetraethyl ammonium). The catholyte 122 may also include divalent cations (e.g., Ca²⁺, Mg²⁺, Zn²⁺) or a divalent cation may be added to the catholyte solution.

A homogenous heterocyclic catalyst 124 is preferably added to the compartment 114b containing the cathode 120. The homogenous heterocyclic catalyst 124 may include, for example, one or more of 4-hydroxy pyridine, adenine, a heterocyclic amine containing sulfur, a heterocyclic amine containing oxygen, an azole, a benzimidazole, a bipyridine, furan, an imidazole, an imidazole related species with at least one five-member ring, an indole, a lutidine, methylimidazole, an oxazole, phenanthroline, pterin, pteridine, a pyridine, a pyridine related species with at least one six-member ring, pyrrole, quinoline, or a thiazole, and mixtures thereof. The homogenous heterocyclic catalyst 124 is preferably present in the compartment 114b at a concentration of between about 0.001M and about 1M, and more preferably between about 0.01M and 0.5M.

The pH of the compartment 114b is preferably between about 1 and 8. A pH range of between about 1 to about 4 is preferable for production of carboxylic acids from carbon dioxide. A pH range of between about 4 to about 8 is preferable for production of carboxylic acid intermediates from carbon dioxide.

The liquid source 104 preferably includes a water source, such that the liquid source 104 may provide pure water to the cell 102. The liquid source 104 may provide other fluids to the cell 102, including an organic solvent, such as methanol, acetonitrile, and dimethylfuran. The liquid source 104 may also provide a mixture of an organic solvent and water to the cell 102.

The energy source 106 may include a variable voltage source. The energy source 106 may be operational to generate an electrical potential between the anode 118 and the cathode 120. The electrical potential may be a DC voltage. In preferred embodiments, the applied electrical potential is generally between about −1.5V vs. SCE and about −4V vs. SCE, preferably from about −1.5V vs. SCE to about −3V vs. SCE, and more preferably from about −1.5 V vs. SCE to about −2.5V vs. SCE.

The gas source 108 preferably includes a carbon dioxide source, such that the gas source 108 may provide carbon dioxide to the cell 102. In some embodiments, the carbon dioxide is bubbled directly into the compartment 114b containing the cathode 120. For instance, the compartment 114b may include a carbon dioxide input, such as a port 126a configured to be coupled between the carbon dioxide source and the cathode 120.

Advantageously, the carbon dioxide may be obtained from any source (e.g., an exhaust stream from fossil-fuel burning power or industrial plants, from geothermal or natural gas wells or the atmosphere itself). Most suitably, the carbon dioxide may be obtained from concentrated point sources of generation prior to being released into the atmosphere. For example, high concentration carbon dioxide sources may frequently accompany natural gas in amounts of 5% to 50%, exist in flue gases of fossil fuel (e.g., coal, natural gas, oil, etc.) burning power plants, and high purity carbon dioxide may be exhausted from cement factories, from fermenters used for industrial fermentation of ethanol, and from the manufacture of fertilizers and refined oil products. Certain geothermal steams may also contain significant amounts of carbon dioxide. The carbon dioxide emissions from varied industries, including geothermal wells, may be captured on-site. Thus, the capture and use of existing atmospheric carbon dioxide in accordance with some embodiments of the present invention generally allow the carbon dioxide to be a renewable and essentially unlimited source of carbon.

The product extractor 110 may include an organic product and/or inorganic product extractor. The product extractor 110 generally facilitates extraction of one or more products (e.g., carboxylic acid, and/or carboxylic acid intermediate) from the electrolyte 122. The extraction may occur via one or more of a solid sorbent, carbon dioxide-assisted solid sorbent, liquid-liquid extraction, nanofiltration, and electrodialysis. The extracted products may be presented through a port 126b of the system 100 for subsequent storage, consumption, and/or processing by other devices and/or processes. For instance, in particular implementations, the carboxylic acid or carboxylic acid intermediate is continuously removed from the cell 102, where cell 102 operates on a continuous basis, such as through a continuous flow-single pass reactor where fresh catholyte and carbon dioxide is fed continuously as the input, and where the output from the reactor is continuously removed. In other preferred implementations, the carboxylic acid or carboxylic acid intermediate is continuously removed from the catholyte 122 via one or more of adsorbing with a solid sorbent, liquid-liquid extraction, and electrodialysis.

The separated carboxylic acid or carboxylic acid intermediate may be placed in contact with a hydrogen stream to produce a glycol or carboxylic acid, respectively. For instance, as shown in FIG. 1B, the system 100 may include a secondary reactor 132 into which the separated carboxylic acid or carboxylic acid intermediate from the product extractor 110 and hydrogen stream from a hydrogen source 134 are introduced. The secondary reactor 132 generally permits interaction between the separated carboxylic acid or carboxylic acid intermediate from the product extractor 110 and the hydrogen to produce a glycol or carboxylic acid, respectively. The secondary reactor 132 may include reactor conditions that differ from ambient conditions. In particular implementations, the secondary reactor 132 preferably includes a temperature range and a pressure range that is higher than that of ambient conditions. For instance, a preferred temperature range of the secondary reactor 132 is between about 50° C. and about 500° C., and a preferred pressure range of the secondary reactor 132 is between about 5 atm and 1000 atm. The secondary reactor may include a solvent and a catalyst to facilitate the reaction between the separated carboxylic acid or carboxylic acid intermediate from the product extractor 110 and the hydrogen stream from the hydrogen source 134. Preferred catalysts include Rh, RuO₂, Ru, Pt, Pd, Re, Cu, Ni, Co, Cu—Ni, and binary metals and/or metal oxides thereof. The catalyst may be a supported catalyst, where the support may include Ti, TiO₂, or C. Preferred solvents include aqueous and non-aqueous solvents, such as water, ether, and tetrahydrofuran.

The oxygen extractor 112 of FIG. 1A is generally operational to extract oxygen (e.g., O₂) byproducts created by the reduction of the carbon dioxide and/or the oxidation of water. In preferred embodiments, the oxygen extractor 112 is a disengager/flash tank. The extracted oxygen may be presented through a port 128 of the system 100 for subsequent storage and/or consumption by other devices and/or processes. Chlorine and/or oxidatively evolved chemicals may also be byproducts in some configurations, such as in an embodiment of processes other than oxygen evolution occurring at the anode 118. Such processes may include chlorine evolution, oxidation of organics to other saleable products, waste water cleanup, and corrosion of a sacrificial anode. Any other excess gases (e.g., hydrogen) created by the reduction of the carbon dioxide and water may be vented from the cell 102 via a port 130.

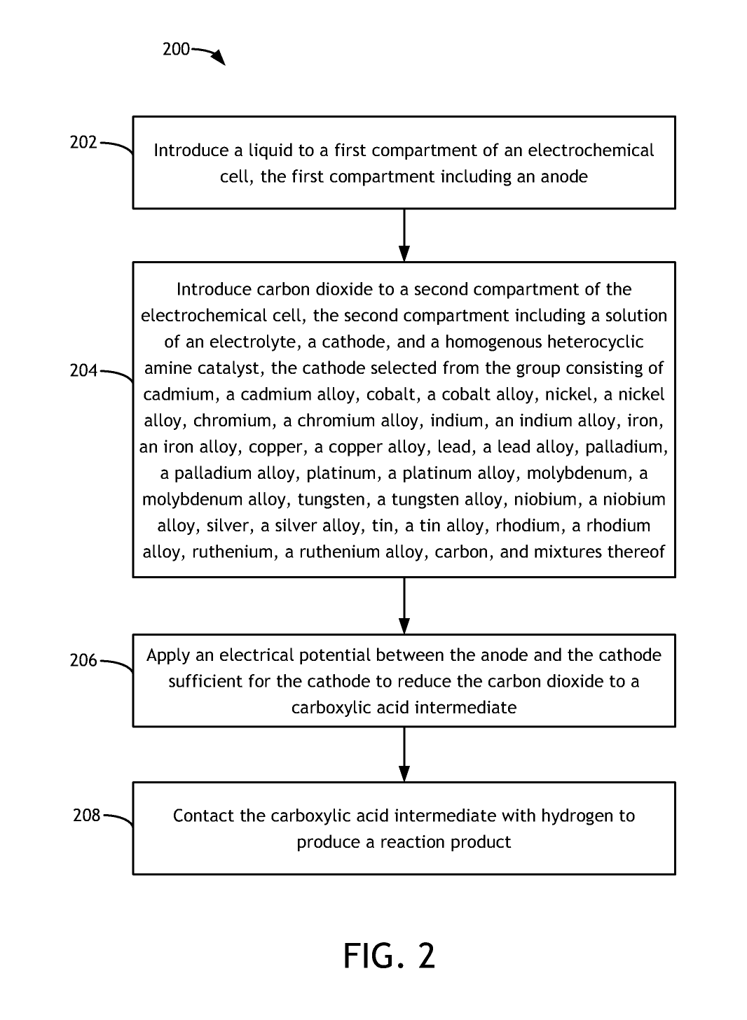

Referring to FIG. 2, a flow diagram of a preferred method 200 for electrochemical conversion of carbon dioxide is shown. The method (or process) 200 generally comprises a step (or block) 202, a step (or block) 204, a step (or block) 206, and a step (or block) 208. The method 200 may be implemented using the system 100.

In the step 202, a liquid may be introduced to a first compartment of an electrochemical cell. The first compartment may include an anode. Introducing carbon dioxide to a second compartment of the electrochemical cell may be performed in the step 204. The second compartment may include a solution of an electrolyte, a cathode, and a homogenous heterocyclic amine catalyst. The cathode may be selected from the group consisting of cadmium, a cadmium alloy, cobalt, a cobalt alloy, nickel, a nickel alloy, chromium, a chromium alloy, indium, an indium alloy, iron, an iron alloy, copper, a copper alloy, lead, a lead alloy, palladium, a palladium alloy, platinum, a platinum alloy, molybdenum, a molybdenum alloy, tungsten, a tungsten alloy, niobium, a niobium alloy, silver, a silver alloy, tin, a tin alloy, rhodium, a rhodium alloy, ruthenium, a ruthenium alloy, carbon, and mixtures thereof. In the step 206, an electric potential may be applied between the anode and the cathode in the electrochemical cell sufficient for the cathode to reduce the carbon dioxide to a carboxylic acid intermediate. The production of the carboxylic acid intermediate is preferably controlled by selection of particular cathode materials, catalysts, pH ranges, and electrolytes, such as disclosed in U.S. application Ser. No. 12/846,221, the disclosure of which is incorporated by reference. Contacting the carboxylic acid intermediate with hydrogen to produce a reaction product may be performed in the step 208. The secondary reactor 132 may permit interaction/contact between the carboxylic acid intermediate and the hydrogen, where the conditions of the secondary reactor 132 may provide for production of particular reaction products.

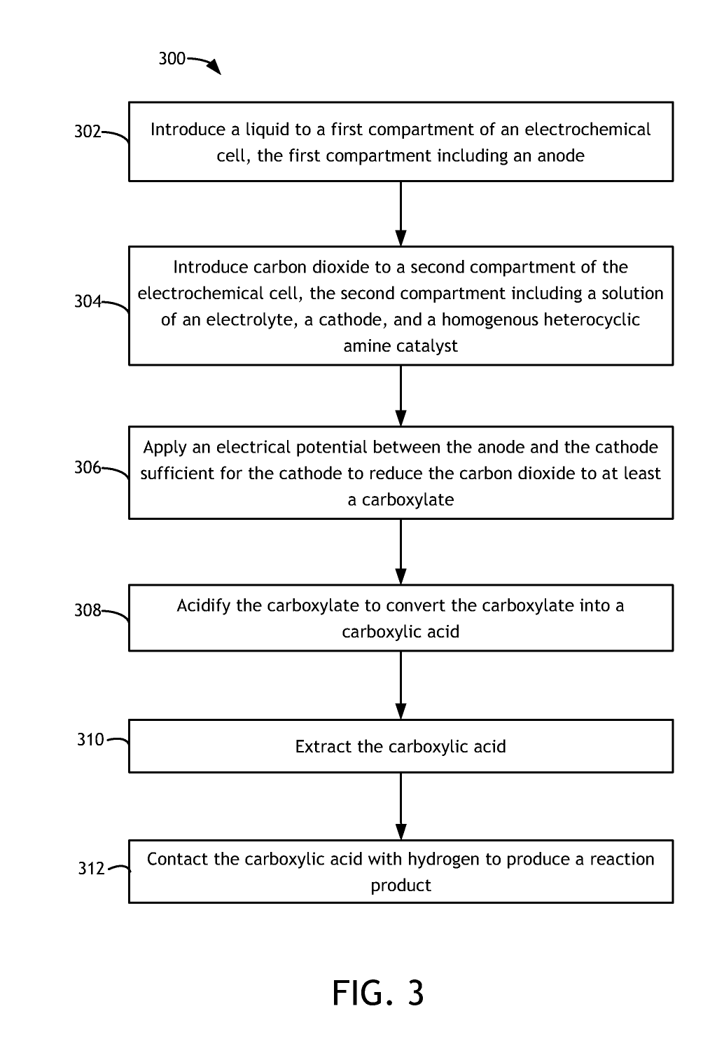

Referring to FIG. 3, a flow diagram of another preferred method 300 for electrochemical conversion of carbon dioxide is shown. The method (or process) 300 generally comprises a step (or block) 302, a step (or block) 304, a step (or block) 306, a step (or block) 308, a step (or block) 310, and a step (or block) 312. The method 300 may be implemented using the system 100.

In the step 302, a liquid may be introduced to a first compartment of an electrochemical cell. The first compartment may include an anode. Introducing carbon dioxide to a second compartment of the electrochemical cell may be performed in the step 304. The second compartment may include a solution of an electrolyte, a cathode, and a homogenous heterocyclic amine catalyst. In the step 306, an electric potential may be applied between the anode and the cathode in the electrochemical cell sufficient for the cathode to reduce the carbon dioxide to at least a carboxylate. Acidifying the carboxylate to convert the carboxylate into a carboxylic acid may be performed in the step 308. The acidifying step may include introduction of an acid from a make-up acid source. In the step 310, the carboxylic acid may be extracted. Contacting the carboxylic acid with hydrogen to form a reaction product may be performed in the step 312. In preferred implementations, the reaction product includes one or more of formaldehyde, methanol, glycolic acid, glyoxal, glyoxylic aid, glycolaldehyde, ethylene glycol, acetic acid, acetaldehyde, ethanol, propylene glycol, or isopropanol.

It is believed that the present disclosure and many of its attendant advantages will be understood by the foregoing description, and it will be apparent that various changes may be made in the form, construction and arrangement of the components thereof without departing from the scope and spirit of the disclosure or without sacrificing all of its material advantages. The form herein before described being merely an explanatory embodiment thereof, it is the intention of the following claims to encompass and include such changes.

What is claimed is:

1. A method for electrochemical conversion of carbon dioxide, comprising:
(A) introducing a liquid to a first compartment of an electrochemical cell, the first compartment including an anode;
(B) introducing carbon dioxide to a second compartment of the electrochemical cell, the second compartment including a solution of an electrolyte, a cathode, and a homogenous heterocyclic amine catalyst, the cathode selected from the group consisting of cadmium, a cadmium alloy, cobalt, a cobalt alloy, nickel, a nickel alloy, chromium, a chromium alloy, indium, an indium alloy, iron, an iron alloy, copper, a copper alloy, lead, a lead alloy, palladium, a palladium alloy, platinum, a platinum alloy, molybdenum, a molybdenum alloy, tungsten, a tungsten alloy, niobium, a niobium alloy, silver, a silver alloy, tin, a tin alloy, rhodium, a rhodium alloy, ruthenium, a ruthenium alloy, carbon, and mixtures thereof;
(C) applying an electrical potential between the anode and the cathode sufficient for the cathode to reduce the carbon dioxide to a carboxylic acid intermediate; and
(D) contacting the carboxylic acid intermediate with hydrogen to produce a reaction product.

2. The method of claim 1, wherein the carboxylic acid intermediate includes at least one of formate, formic acid, glycolate, glycolic acid, glyoxylate, glyoxylic acid, lactate, lactic acid, oxalate, or oxalic acid.

3. The method of claim 1, wherein the reaction product includes at least one of formaldehyde, formic acid, methanol, glyoxylic acid, glycolic acid, glyoxal, glycolaldehyde, ethylene glycol, acetic acid, acetaldehyde, ethanol, lactic acid, oxalic acid, propylene glycol, or isopropanol.

4. The method of claim 1, wherein the carboxylic acid intermediate includes formic acid, and wherein the reaction product includes at least one of formaldehyde or methanol.

5. The method of claim 1, wherein the carboxylic acid intermediate includes oxalic acid, and wherein the reaction product includes at least one of glyoxylic acid, glycolic acid, glyoxal, glycolaldehyde, ethylene glycol, acetic acid, acetaldehyde, or ethanol.

6. The method of claim 1, wherein the carboxylic acid intermediate includes lactic acid, and wherein the reaction product includes at least one of propylene glycol or isopropanol.

7. The method of claim 1, wherein the carboxylic acid intermediate includes glyoxylic acid, and wherein the reaction product includes at least one of glycolic acid, glyoxal, glycolaldehyde, ethylene glycol, acetic acid, acetaldehyde, or ethanol.

8. The method of claim 1, wherein the carboxylic acid intermediate includes glycolic acid, and wherein the reaction product includes at least one of glycolaldehyde, ethylene glycol, acetic acid, acetaldehyde, or ethanol.

9. The method of claim 1, wherein a pH of the second compartment is between about 1 and about 8.

10. The method of claim 1, further comprising:
adjusting a pH of the second compartment to favor production of one of a carboxylic acid and a carboxylic acid intermediate over production of the other of the one of a carboxylic acid and a carboxylic acid intermediate.

11. A system for electrochemical reduction of carbon dioxide, comprising:
an electrochemical cell including:
a first cell compartment;
an anode positioned within said first cell compartment;
a second cell compartment;
a separator interposed between said first cell compartment and said second cell compartment, said second cell compartment containing an electrolyte; and
a cathode and a homogenous heterocyclic amine catalyst positioned within said second cell compartment, said cathode selected from the group consisting of cadmium, a cadmium alloy, cobalt, a cobalt alloy, nickel, a nickel alloy, chromium, a chromium alloy, indium, an indium alloy, iron, an iron alloy, copper, a copper alloy, lead, a lead alloy, palladium, a palladium alloy, platinum, a platinum alloy, molybdenum, a molybdenum alloy, tungsten, a tungsten alloy, niobium, a niobium alloy, silver, a silver alloy, tin, a tin alloy, rhodium, a rhodium alloy, ruthenium, a ruthenium alloy, carbon, and mixtures thereof;
an energy source operably coupled with said anode and said cathode, said energy source configured to apply a voltage between said anode and said cathode to reduce carbon dioxide at said cathode to an intermediate product stream including a carboxylic acid;
an extractor configured to extract the carboxylic acid from the intermediate product stream; and
a secondary reactor configured to introduce the carboxylic acid to hydrogen from a hydrogen source, the secondary reactor configured to produce at least one of formaldehyde, methanol, glycolic acid, glyoxal, glyoxylic acid, glycolaldehyde, ethylene glycol, acetic acid, acetaldehyde, ethanol, propylene glycol, or isopropanol.

12. A method for electrochemical conversion of carbon dioxide, comprising:
(A) introducing a liquid to a first compartment of an electrochemical cell, the first compartment including an anode;
(B) introducing carbon dioxide to a second compartment of the electrochemical cell, the second compartment including a solution of an electrolyte, a cathode, and a homogenous heterocyclic amine catalyst;
(C) applying an electrical potential between the anode and the cathode sufficient for the cathode to reduce the carbon dioxide to at least a carboxylate;
(D) acidifying the carboxylate to convert the carboxylate into a carboxylic acid;
(E) extracting the carboxylic acid; and
(F) contacting the carboxylic acid with hydrogen to form a reaction product.

13. The method of claim 12, wherein the carboxylate includes at least one of formate, glycolate, glyoxylate, lactate, or oxalate.

14. The method of claim 12, wherein the carboxylic acid includes at least one of formic acid, glycolic acid, glyoxylic acid, lactic acid, or oxalic acid.

15. The method of claim 12, wherein the reaction product includes at least one of formaldehyde, methanol, glycolic acid, glyoxal, glyoxylic aid, glycolaldehyde, ethylene glycol, acetic acid, acetaldehyde, ethanol, propylene glycol, or isopropanol.

16. The method of claim 12, wherein the carboxylate includes formate, the carboxylic acid intermediate includes formic acid, and the reaction product includes at least one of formaldehyde or methanol.

17. The method of claim 12, wherein the carboxylate includes oxalate, the carboxylic acid intermediate includes oxalic acid, and the reaction product includes at least one of glyoxylic acid, glycolic acid, glyoxal, glycolaldehyde, ethylene glycol, acetic acid, acetaldehyde, or ethanol.

18. The method of claim 12, wherein the carboxylate includes lactate, the carboxylic acid intermediate includes lactic acid, and the reaction product includes at least one of propylene glycol or isopropanol.

19. The method of claim 12, wherein the carboxylate includes glycolate, the carboxylic acid intermediate includes glycolic acid, and the reaction product includes at least one of glycolaldehyde, ethylene glycol, acetic acid, acetaldehyde, or ethanol.

20. The method of claim 12, wherein the carboxylate includes glyoxylate, the carboxylic acid intermediate includes glyoxylic acid, and the reaction product includes at least one of glycolic acid, glyoxal, glycolaldehyde, ethylene glycol, acetic acid, acetaldehyde, or ethanol.
